(19) United States
(12) Patent Application Publication
(10) Pub. No.: US 2005/0238605 A1
(43) Pub. Date: Oct. 27, 2005

(54) Low-viscosity O/W emulsion

(75) Inventors: Silke Kohlhase, Hamburg (DE); Andreas Schafer, Hamburg (DE); Ingo Hahn, Pinneberg (DE); Celina Storbeck, Bonningstedt (DE); Susanne Morgenroth, Norderstedt (DE); Ulrich Hain, Hamburg (DE)

Correspondence Address: ALSTON & BIRD LLP;BANK OF AMERICA PLAZA, 101 SOUTH TRYON STREET, SUITE 4000, CHARLOTTE, NC, 28280-4000, US

(73) Assignee: Beiersdorf AG

(21) Appl. No.: 11/041,016

(22) Filed: Jan. 21, 2005

(30) Foreign Application Priority Data
Jan. 21, 2004 (DE) 10 2004 003 435.4

(51) Int. Cl. A61K 7/06; A61K 7/11

(52) U.S. Cl. 424/70.13; 424/70.16

(57) ABSTRACT

Cosmetic and/or dermatological oil-in-water (O/W) emulsion with a viscosity of 10 to 1500 mPaS, containing a) one or more emulsifiers selected from the group of glyceryl stearate citrate, polyglycerol methyl glucose distearate, triceteareth-4-phosphate, b) a homopolymer or copolymer of the acrylic acid and/or its derivatives as polymeric stabilizer, wherein the total concentration of the sum of emulsifier a) and polymeric stabilizer b) is between 0.01% and 1.5% by weight based on the total weight of the emulsion, and wherein the emulsion is free from fatty alcohols with a chain length of 12 to 20 carbon atoms.

The present invention relates to a low-viscosity oil-in-water (O/W) emulsion.

For years, the production of cosmetic cleansing agents has shown an increasing trend. This results primarily from an increasing health consciousness and need for hygiene of the consumers.

Cleansing of the human body means removal of (environmental) dirt and, with that, causes an increase of psychic and physical wellbeing. Cleansing of the surface of skin and hair is a very complex procedure that is dependent on many parameters. On the one hand, the procedure is intended to remove as completely as possible exogenous substances, such as, for example, hydrocarbons or inorganic pigments from a great variety of ambient fields, as well as residues from cosmetics, or also undesired microorganisms. On the other, it is intended to wash off endogenous excretions, such as perspiration, sebum, skin scales, and dandruff, without deep-reaching interventions into the physiological balance of the skin.

To cleanse skin and skin appendages, it is common to use emulsions in the form of cleansing emulsions in addition to surfactant-containing cleansing preparations. They are used in most cases for cleansing the face and in particular for removing decorative cosmetics.

Contrary to other cleansing preparations, such as, for example, soap, cleansing emulsions have the advantage of being well tolerated by the skin, since they may contain in their lipophilic phase skin-caring oils and nonpolar active ingredients, such as vitamin E.

A particularly common form of cosmetic emulsions are low-viscosity oil-in-water emulsions (O/W emulsions). Low-viscosity O/W emulsions are normally based on an emulsifier system of ceteareth-20, ceteareth 12, cetearyl alcohol, cetyl palmitate, and glyceryl stearate. Further O/W emulsifier systems are: triceteareth-4 phosphate; trilaureth-4 phosphate; glyceryl isostearate+isoceteth-20; PEG-60 hydrogenated castor oil, or polysorbate 20.

However, in the art, preparations of this type have the disadvantage that last but not least because of their relatively high emulsifier content, they leave a sticky sensation on the skin and offer little care. Furthermore, such preparations normally have a far too short shelf life (long-term stability).

Furthermore, highly viscous O/W emulsions with polyethylene glycol esters as emulsifiers are known in the art. Emulsions with polyethylene glycol esters as emulsifiers have the advantage that they are long-term stable and electrolyte compatible. Furthermore, they leave a rich, nonsticky sensation on the skin, and are well tolerated by the skin.

However, preparations of this type have the disadvantage that they cover only a limited application spectrum because of their viscosity. They can neither be sprayed, nor are they suitable as impregnation medium for wipes.

To solve the problems of the art, different approaches have been taken in the past. For example, Pickering pigments were used as emulsifiers (DE 102 38 649.8). A further approach consisted in using a high concentration of emulsifier (2% by weight and more) in combination with fatty alcohols (DE 198 02 206 and DE 101 29 973) or W/O emulsions (DE 101 54 627).

A special product form for cleansing preparations are solid cleansing substrates or cleansing textiles, in particular wipes. These may be impregnated already by the manufacturer with the cleansing preparation (a combination, which is also named “cleansing article” within the scope of the present invention), and thus have the advantage that they already contain the preparation in the correct dosage. Furthermore, they avoid the disadvantage of preparations stored in bottles, whose package may break and whose content may “leak.” Further advantages of cleansing substrates/textiles also include the situation that they are easy to take along on trips in counted quantities, and normally do no longer need water for their application. Cleansing substrates/textiles are made from textiles. Textiles may be woven or knit, and be present as composite material (nonwoven textile). In most cases, composite materials are used (for cost reasons). In the case of composite materials, the fabric is not produced by warp and weft or stitch formation, but by interlocking, and/or cohesive and/or adhesive bonding of textile fibers. According to DIN 61210 T2, composite materials may be distinguished by nonwoven webs, paper, batting, and felt. Nonwoven webs are loose materials produced from spun fibers (i.e. fibers of a defined length), or filaments (endless fibers), in most cases of polypropylene, polyester, or rayon. Typically, their cohesion is provided by the fibers holding together. In this connection, the individual fibers may have a preferred orientation (oriented or cross-laid webs), or be unoriented (entangled webs). The fibers may be mechanically bonded by needle punching, stitching, or entangling by means of strong water jets. Adhesively bonded nonwoven fabrics are produced by gluing the fibers together with liquid binding agents (for example, acrylate polymers, SBR/NBR, polyvinyl ester, polyurethane dispersions), or by melting or dissolving so-called binder fibers that are added to the web during its production. In the case of cohesive bonding, the fiber surfaces are partially dissolved by suitable chemicals and bonded by pressure or fused at an increased temperature [J. Falbe, M. Regnitz: Römpp-Chemie-Lexikon, 9^{th} edition, Thieme-Verlag, Stuttgart (1992)].

Substrates that are impregnated with cosmetic preparations and in particular wipes may be produced in different ways: in a so-called “dip method”, the wipe is immersed into a dip bath or pulled through a bath. This method is especially suited for paper wipes and less suited for nonwoven fabrics, since the latter absorb too much liquid (=preparation), and when being repackaged, subsequently find themselves in puddles of the preparation that has been released again.

A second variant is the “spraying method”, wherein the preparation is sprayed onto the advancing fabric. While this method is suitable for all textiles, it does not permit applying heavily foaming preparations, since the foam development becomes too great in the spraying method.

As further methods, so-called squeegee methods are used, wherein nonwoven or fabric webs advance along doctor blades, doctor bars, or doctor nozzles, which continuously receive an impregnation solution. Different degrees of impregnation may be adjusted, among other things, by varying the contact pressure and the rate of advance of the fabric.

However, substrates/fabrics as produced in the art, which are impregnated with cleansing preparations or other cosmetic preparations (cleansing articles) have a series of disadvantages: For example, substrates/fabrics that are treated with conventional impregnation media are characterized by an aqueous/empty sensory characteristic. They have no more than an unsatisfactory cleansing efficiency, in particular for waterproof cosmetic products (for example, mascaras, etc.). Moreover, such products are characterized by just a limited shelf life, which makes itself known in particular by a draining of the products. This leads to unevenly impregnated products, which the consumer finds qualitatively inferior. Furthermore, conventional fabric products are only hard to preserve in a consumer-satisfactory and long-term stable manner.

It was therefore an object of the present invention to eliminate the disadvantages of the art. In particular, it was an object to develop low-viscosity emulsions as well as cleansing textiles, which avoid the deficiencies of the art. It has furthermore been an object of the present invention to develop low-viscosity, particularly storage stable O/W emulsions, which leave at most a minimally sticky sensation on the skin and are excellently tolerated by the skin.

Last but not least, the O/W emulsion is to meet the requirement of being both suitable as an impregnating formulation for fabrics and other textiles, and sprayable by means of a sprayer (for example, an aerosol can, pump spray).

Surprisingly, these objects are accomplished by a cosmetic and/or dermatological oil-in-water (O/W) emulsion with a viscosity of 10 to 1500 mPas, containing
a) one or more emulsifiers selected from the group of glyceryl stearate citrate, polyglycerol methyl glucose distearate, triceteareth-4-phosphate;
b) a homopolymer or copolymer of the acrylic acid and/or its derivatives as polymeric stabilizer; wherein the total concentration of the sum of emulsifier a) and polymeric stabilizer b) is between 0.01 and 1.5% by weight based on the total weight of the emulsion, and the emulsion is free from fatty alcohols with a chain length of 12 to 20 carbon atoms.

The viscosity values of the preparations and individual substances as specified within the scope of the present Application, were determined with the aid of a viscosity meter of the type Viskotester VT 02 by Haake (temperature: 25° C., 23 mm spindle diameter, 62.5 rpm rotor speed).

In accordance with the invention, it is advantageous to use glyceryl stearate citrate as emulsifier a). Glyceryl stearate citrate (CAS 39175-72-9, INCI: glyceryl stearate citrate) can be obtained, inter alia, under the trade name Imwitor from the manufacturer Hüls. In accordance with the invention, it is advantageous to use glyceryl stearate citrate in a concentration from 0.01% to 1.5% by weight, and preferably in a concentration from 0.1% to 1.3% by weight, each based on the total weight of the preparation.

In a further advantageous form of realization of the present invention, one uses as emulsifier a) polyglycerol methyl glucose distearate. Polyglycerol methyl glucose distearate (INCI: polyglycerol methyl glucose distearate) can be obtained, inter alia, under the trade name Tego Care-450 from the manufacturer Goldschmidt. According to the invention, it is advantageous to use polyglycerol methyl glucose distearate in a concentration from 0.01% to 1.5% by weight, preferably in a concentration from 0.1% to 1.3% by weight, each based on the total weight of the preparation.

In a further advantageous form of realization of the present invention, triceteareth-4-phosphate is used as emulsifier a). Triceteareth-4-phosphate (INCI: triceteareth-4-phosphate) is commercially available, for example, from the manufacturer Clariant under the trade name Hostaphat KW 340D. According to the invention, it is advantageous to use triceteareth-4-phosphate in a concentration from 0.01% to 1.5% by weight, and preferably in a concentration from 0.1% to 1.3% by weight, each based on the total weight of the preparation.

In the emulsions of the invention, it is possible to use polymeric stabilizers. Advantageous according to the invention are, for example, homo- or copolymers of the acrylic acid.

Examples to name include compounds originating from group of the so-called carbomers or carbopols (Carbopol® is actually a registered trademark of B.F. Goodrich Company). The advantageous acrylate-alkylacrylate copolymers according to the invention are characterized by the following structure 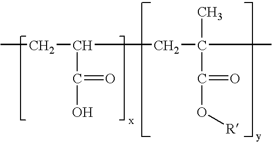
where R′ is a long-chain alkyl residue and x and y are numbers that symbolize the respective stoichiometric component of the respective comonomers.

Preferred in accordance with the invention are acrylate copolymers and/or acrylate-alkylacrylate copolymers, which are available, for example, under the trade names Carbopol® 1382, Carbopol® 981, and Carbopol 5984® from B.F. Goodrich Company, as well as polyacrylates from the group of the carbopols of the grades 980, 981, 1382, 2984, 5984, as well as carbomer 2001. Also advantageous is sodium polyacrylate, which is available, for example, under the trade name Cosmedia SP from Cognis.

Also advantageous are copolymers/cross polymers that comprise sodium acrylate, such as Aqua SF-1 from Noveon.

Also advantageous are copolymers from C₁₀₋₃₀-alkylacrylates and one or more monomers of the acrylic acid, methacrylic acid, or their esters, which are crosslinked with an allyl ether of saccharose or an allyl ether of pentaerythritol.

Especially advantageous are compounds, which bear the INCI description “acrylate/C₁₀₋₃₀ alkyl acrylate cross polymer.” Particularly advantageous are those which are available under the trade names Permulen TR1 and Permulen TR2, Carbopol 1328, Ultrez 21 from B.F. Goodrich Company, as well as under the tradename Tegocarbomer TC 341 ER from Goldschmidt/Degussa.

Also advantageous are AMPS polymers and copolymers (AMPS: acrylamidomethylpropyl sulfonic acid), such as, for, example, the polymer, which is filed under the Chemical Abstracts registration numbers 58374-69-9, 13162-05-5, and 88-12-0 and available under the trade name Aristoflex® AVC from Clariant GmbH (INCI: ammonium acryloyldimethyl taurate/vinylpyrrolidone copolymers).

In accordance with the invention, the advantageous ammonium acryloyldimethyl taurate/vinyl pyrrolidone copolymer has the empirical formula [C₇H₁₆N₂SO₄]ₙ[C₆H₉NO]ₙ corresponding to a statistic structure as follows:

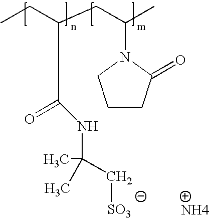

Also advantageous are copolymers/cross polymers comprising acryloyldimethyl taurate, such as, for example, Simugel® EG or Simugel® EG from Seppic S.A.

Unless previously preneutralized, it is common to neutralize the aforesaid synthetic polyelectrolytes at least in part by adding suitable inorganic or organic bases, such as, for example, sodium hydroxide solution, potassium hydroxide solution, triethyl amine, or ammonia water, and to thus convert them into the advantageoue ionic salt form for the purposes of the application.

In accordance with the invention, it is advantageous, when the emulsion of the invention is characterized in that the concentration of polymeric stabilizers is from 0.001% to 1.5% by weight, and preferably 0.05% to 0.2% by weight based on the total weight of the preparation.

According to the invention, the weight ratio of emulsifier a) to polymeric stabilizer b) advantageously ranges from 20:1 to 1:1.

The emulsions of the invention may advantageously contain in accordance with the invention the following constituents:

In accordance with the invention, the emulsions of the invention may advantageously contain as aqueous phase besides water also other ingredients, for example, alcohols, diols or polyols of a low number of carbons, as well as ethers thereof, preferably ethanol, isopropanol, propylene glycol, glycerol, ethylene glycol, ethylene glycol monoethyl- or -monobutyl ether, propylene glycol monomethyl, -monoethyl-, or -monobutyl ether, diethylene glycol monomethyl or -monoethyl ether, di ethylene glycol monomethyl- or -monoethyl ether, and analogous products, furthermore alcohols of a low carbon number, for example, ethanol, isopropanol, 1,2-propanediol, and glycerol.

The emulsion of the invention contains one or more oil, lipid, and/or wax components of identical or different polarity. Polar oils are, for example, those from the group of the lecithins and fatty acid triglycerides, namely the triglycerol esters of saturated and/or unsaturated, branched and/or unbranched alkane carboxylic acids having a chain length from 8 to 24, in particular 12 to 18 carbon atoms. The fatty acid triglycerides may advantageously be selected, for example, from the group of the synthetic, semisynthetic, and natural oils, for example, olive oil, sunflower seed oil, soy oil, peanut oil, rape oil, almond oil, palm oil, coconut oil, castor oil, wheat germ oil, grape seed oil, thistle oil, evening primrose oil, macadamia oil, and more of the like.

Particularly advantageous polar lipids for the purposes of the present invention are all native lipids, such as, for example, olive oil, sunflower seed oil, soy oil, peanut oil, rape oil, almond oil, palm oil, coconut oil, castor oil, wheat germ oil, grape seed oil, thistle oil, evening primrose oil, macadamia oil, corn oil, avocado oil, and the like, as well as those listed in the following.

| | | | Polarity |
|---|---|---|---|
| Manufacturer | Trade Name | INCI-Name | [mN/m] |
| Condea Chemie | Isofol 14 T | Butyl decanol (+) hexyl octanol (+) | 19.8 |
| | | hexyl Decanol (+) butyl octanol | |
| Lipochemicals | Lipovol MOS-130 | Tridecyl stearate (+) tridecyl | 19.4 |
| INC./USA | | trimellitate(+) dipentaerythrityl | |
| (Induchem) | | hexacaprylate/hexacaprate | |
| | Castor oil | | 19.2 |
| CONDEA Chemie | Isofol Ester 0604 | | 19.1 |
| Huels | Miglyol 840 | Propylene glycol | 18.7 |
| CONDEA Chemie | | dicaprylate/dicaprate | |
| CONDEA | Isofol 12 | Butyl octanol | 17.4 |
| Chemie | | | |
| Goldschmidt | Tegosoft SH | Stearyl heptanoate | 17.8 |
| | Avocado oil | | 14.5 |
| Henkel Cognis | Cetiol B | Dibutyl adipate | 14.3 |
| ALZO (ROVI) | Dermol 488 | PEG 2 diethylenhexanoate | 10.1 |
| Condea Augusta | Cosmacol ELI | C12-13 Alkyl lactate | 8.8 |
| S.P.A. | | | |
| ALZO (ROVI) | Dermol 489 | Diethylene glycol dioctanoate/ | 8.6 |
| | | diisononanoate | |
| Condea Augusta | Cosmacol ETI | Di-C12/13 alkyl tartrate | 7.1 |
| S.P.A. | | | |
| Henkel Cognis | Emerest 2384 | Propylene glycol monoisostearate | 6.2 |
| Henkel Cognis | Myritol 331 | Cocoglycerides | 5.1 |
| Unichema | Prisorine 2041 | Triisostearin | 2.4 |
| | GTIS | | |

Furthermore, the oil phase may advantageously be selected from the group of the dialkyl ethers. It will be particularly advantageous, when the oil phase has a content of C₁₂₋₁₅ alkyl benzoate or totally consists of same.

Furthermore, the oil phase may advantageously be selected from the group of Guerbet alcohols. Guerbet alcohols are named after Marcel Guerbet, who described their production for the first time. They form according to the reaction equation 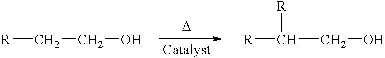
by oxidizing an alcohol to an aldehyde, by aldol condensation of the aldehyde, separation of water from the aldol, and hydrogenation of the allyl aldehyde. Guerbet alcohols are liquid even at low temperatures, and practically cause no skin irritations. It is possible and advantageous to use them as fatting, superfatting, and also as refatting active ingredients in skin care and hair care agents.

The use of Guerbet alcohols in cosmetics is known per se. In most cases, species of this type are characterized by the structure 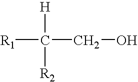
where R₁ and R₂ are normally unbranched alkyl residues.

In accordance with the invention, the Guerbet alcohol or alcohols is/are selected from the group, where
R₁=propyl, butyl, pentyl, hexyl, heptyl, or octyl; and
R₂=hexyl, heptyl, octyl, nonyl, decyl, undecyl, dodecyl, tridecyl, or tetradecyl.

Preferred Guerbet alcohols in accordance with the invention are 2-butyloctanol—it has the chemical structure 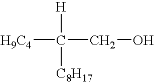
and is available, for example, under the trade name Isofol® 12 from Condea Chemie GmbH—and 2-hexyldecanol—it has the chemical structure 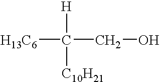
and is available, for example, under the trade name Isofol® 16 from Condea Chemie GmbH. Likewise mixtures of Guerbet alcohols in accordance with the invention are advantageous to use in accordance with the invention. Mixtures of 2-butyloctanol and 2-hexyldecanol are available, for example, under the trade name Isofol® 14 from Condea Chemie GmbH.

The total quantity of Guerbet alcohols in ready-to-use cosmetic or dermatological preparations is advantageously selected from the range up to 25% by weight, preferably 0.5 to 15.0% by weight based on the total weight of the preparations.

It is likewise advantageous to use any mixtures of such oil and wax components for the purposes of the present invention. If need be, it may also be advantageous to use waxes, for example, cetyl palmitate as the only lipid component of the oil phase.

Particularly advantageous medium-polarity lipids for the purposes of the present invention are the substances listed below:

| | | | Polarity |
|---|---|---|---|
| Manufacturer | Trade Name | INCI-Name | [mN/m] |
| Stearinerie Dubois | DUB VCI 10 | Isodecyl neopentanoate | 29.9 |
| Fils | | | |
| ALZO (ROVI) | Dermol IHD | Isohexyl decanoate | 29.7 |
| ALZO (ROVI) | Dermol 108 | Isodecyl octanoate | 29.6 |
| | Dihexyl ether | Dihexyl ether | 29.2 |
| ALZO (ROVI) | Dermol 109 | Isodecyl 3,5,5 trimethyl hexanoate | 29.1 |
| Henkel Cognis | Cetiol SN | Cetearyl isononanoate | 28.6 |
| Unichema | Isopropyl palmitate | Isopropyl palmitate | 28.8 |
| Dow Corning | DC Fluid 345 | Cyclomethicone | 28.5 |
| Dow Corning | Dow Corning Fluid | Cyclopolydimethylsiloxane | 28.5 |
| | 244 | | |
| Nikko Chemicals | Jojoba Oil Gold | | 26.2 |
| Superior Jojoba | | | |
| Oil Gold | | | |
| Wacker | Wacker AK 100 | Dimethicone | 26.9 |
| ALZO (ROVI) | Dermol 98 | 2-Ethylhexanoic acid 3,5,5 trimethyl | 26.2 |
| | | ester | |
| Dow Corning | Dow Corning Fluid | Open | 25.3 |
| | 246 | | |
| ALZO (ROVI) | Dermol 139 | Isotridecyl-3,5,5-trimethyl | 24.5 |
| | | hexanonanoate | |
| | Cegesoft C24 | Octyl palmitate | 23.1 |
| Gattefossé | M.O.D. | Octyldodeceyl myristate | 22.1 |
| | Macadamia Nut | | 22.1 |
| | Oil | | |
| Bayer AG, | Silikonöl VP 1120 | Phenyl trimethicone | 22.7 |
| Dow Corning | | | |
| CONDEA Chemie | Isocarb 12 | Butyl octanoic acid | 22.1 |
| Henkel Cognis | Isopropyl stearate | Isopropyl stearate | 21.9 |
| WITCO, | Finsolv TN | C12-15 Alkyl benzoate | 21.8 |
| Goldschmidt | | | |
| Dr. Straetmans | Dermofeel BGC | Butylene glycol caprylate/caprate | 21.5 |
| Unichema | Miglyol 812 | Caprylic/capric triglyceride | 21.3 |
| Huels | | | |
| Trivent (via S. | Trivent OCG | Tricaprylin | 20.2 |
| Black) | | | |
| ALZO (ROVI) | Dermol 866 | PEG,, Diethyl hexanoate/ | 20.1 |
| | | diisononanoate/ethylhexyl | |
| | | isononanoate | |

Nonpolar oils are, for example, those which are selected from the group of branched and unbranched hydrocarbons and hydrocarbon waxes, in particular Vaseline (petrolatum) paraffin oil, squalane and squalene, polyolefins, and hydrogenated polyisobutenes. Among the polyolefins, the preferred substances are polydecenes.

Particularly advantageous nonpolar lipids for the purposes of the present invention are the substances of the following list:

| | | | Polarity |
|---|---|---|---|
| Manufacturer | Trade Name | INCI-Name | [mN/m] |
| Total SA | Ecolane 130 | Cycloparaffin | 49.1 |
| Neste PAO N.V. | Nexbase 2006 FG | Polydecene | 46.7 |
| (supplier Hansen & Rosenthal) | | | |
| Chemische Fabrik Lehrte | Polysynlane | Hydrogenated | 44.7 |
| | | polyisobutene | |
| Wacker | Wacker Silcone Oil | Polydimethyl siloxane | 46.5 |
| | AK 50 | | |
| EC Erdölchemie (supplier Bayer AG) | Solvent ICH | Isohexadecane | 43.8 |
| DEA Mineralöl (supplier Hansen & | Pionier 2076 | Mineral oil | 43.7 |
| Rosenthal) | | | |
| Tudapetrol | | | |
| DEA Mineralöl (supplier Hansen & | Pionier 6301 | Mineral oil | 43.7 |
| Rosenthal) | | | |
| Tudapetrol | | | |
| Wacker | Wacker Silicone Oil | Polydimethyl siloxane | 42.4 |
| | AK 35 | | |
| EC Erdölchemie GmbH | Isoeicosane | Isoeicosane | 41.9 |
| Wacker | Wacker Silicone Oil | Polydimethylsiloxane | 40.9 |
| | AK 20 | | |
| Condea Chemie | Isofol 1212 | | 40.3 |
| | Carbonate | | |
| Gattefossé | Softcutol O | Ethoxydiglycol oleate | 40.5 |
| Creaderm | Lipodermanol OL | Decyl olivate | 40.3 |
| Henkel | Cetiol S | Dioctylcyclohexane | 39.0 |
| DEA Mineralöl (supplier Hansen & | Pionier 2071 | Mineral oil | 38.3 |
| Rosenthal) | | | |
| Tudapetrol | | | |
| WITCO BV | Hydrobrite 1000 PO | Paraffinum liquidum | 37.6 |
| Goldschmidt | Tegosoft HP | Isocetyl palmitate | 36.2 |
| Condea Chemie | Isofol Ester 1693 | | 33.5 |
| Condea Chemie | Isofol Ester 1260 | | 330 |
| Dow Corning | Dow Corning Fluid | Cyclopentasiloxane | 32.3 |
| | 245 | | |
| Unichema | Prisorine 2036 | Octyl Isostearate | 31.6 |
| Henkel Cognis | Cetiol CC | Dicaprylyl carbonate | 31.7 |
| ALZO (ROVI) | Dermol 99 | Trimethylhexyl | 31.1 |
| | | isononanoate | |
| ALZO (ROVI) | Dermol 89 | 2-Ethylhexyl | 31.0 |
| | | Isononanoate | |
| Henkel Cognis | Cetiol OE | Dicaprylyl ether | 30.9 |
| | Dihexylcarbonate | Dihexyl carbonate | 30.9 |
| Albemarle S.A. | Silkflo 366 NF | Polydecene | 30.1 |
| Unichema | Estol 1540 EHC | Octyl cocoate | 30.0 |

However, it is also advantageous to use mixtures of lipids having a higher and lower polarity and the like. Thus, the oil phase may advantageously be selected from the group of branched and unbranched hydrocarbons and hydrocarbon waxes, the dialkyl ethers, the group of the saturated or unsaturated, branched or unbranched alcohols, as well as the fatty acid triglycerides, in particular the triesters of glycerin of saturated and/or unsaturated, branched and/or unbranched alkanoic acids having a chain length of 8 to 24, in particular 12-18 carbon atoms. The fatty acid triglycerides may advantageously be selected from the group of the synthetic, semisynthetic, and natural oils, olive oil, sunflower seed oil, soybean oil, peanut oil, rape seed oil, almond oil, palm oil, coconut oil, palm kernel oil, and more of the like, provided the conditions of claim 1 are met.

Fat and/or wax components that are advantageous to use in accordance with the invention may be selected from the group of vegetable waxes, animal waxes, mineral waxes and petrochemical waxes. Examples which are favorable according to the invention are candelilla wax, carnauba wax, Japan wax, esparto grass wax, cork wax, guaruma wax, rice germ oil wax, sugarcane wax, berry wax, ouricury wax, montan wax, jojoba wax, shea butter, beeswax, shellac wax, spermaceti, lanolin (wool wax), uropygial grease, ceresin, ozocerite (earth wax), paraffin waxes, and microcrystalline waxes, provided the conditions of claim 1 are met.

Other advantageous fat and/or wax components are chemically modified waxes and synthetic waxes such as, for example, those obtainable under the trade names Syncrowax HRC (glyceryl tribehenate), and Syncrowax AW 1C (C₁₈₋₃₆-fatty acid) from CRODA GmbH, and also montan ester waxes, Sasol waxes; hydrogenated jojoba waxes, synthetic or modified beeswaxes (e.g. dimethicone copolyol beeswax and/or C₃₀₋₅₀-alkyl beeswax), polyalkylene waxes, polyethylene glycol waxes, but also chemically modified fats, such as, for example, hydrogenated vegetable oils (for example, hydrogenated castor oil and/or hydrogenated coconut oil glycerides), triglycerides, such as, for example, trihydroxy stearin, fatty acids, fatty acid esters and glycol esters, such as, for example, C₂₀₋₄₀-alkyl stearate, C₂₀₋₄₀-alkylhydroxystearoyl stearate and/or glycol montanate. Also advantageous are certain organosilicon compounds, which have physical properties similar to the specified fatty and/or wax components, such as, for example, stearoxytrimethylsilane, provided the conditions of claim 1 are met.

In accordance with the invention, the fat and/or wax components may be present both individually and as a mixture. Likewise any desired mixtures of such oil and wax components may be advantageously used for the purposes of the present invention.

Advantageously, the oil phase is selected from the group of 2-ethylhexyl isostearate, octyldodecanol, isotridecyl isononanoate, butylene glycol, dicaprylate/dicarpate, 2-ethylhexyl cocoate, C₁₂₋₁₅-alkyl benzoate, caprilic-capric acid triglyceride, dicaprylyl ether, provided the conditions of claim 1 are met.

Especially advantageous are mixtures of octyldodecanol, carpylic-capric acid triglyceride, dicaprylyl ether, dicaprylyl carbonate, coconut oil glycerides, or mixtures of C₁₂₋₁₅-alkyl benzoate and 2-ethylhexyl isostearate, mixtures of C₁₂₋₁₅-alkyl benzoate and butylene glycol dicaprylate/dicaprate, as well as mixtures of C₁₂₋₁₅-alkyl benzoate, 2-ethylhexyl isostearate, and isotridecyl isononanoate, provided the conditions of claim 1 are met.

Of the hydrocarbons, paraffin oil, cycloparaffin, squalane, squalene, hydrogenated polyisobutene, or polydecene are advantageous to use for the purposes of the present invention, provided the conditions of claim 1 are met.

Silicones

It is also possible and advantageous to select the oil phase of the preparations according to the invention in part or in full from the group of cyclic and/or linear silicones, which are also named “silicone oils” within the scope of the present disclosure. Such silicones or silicone oils may be present as monomers, which are normally characterized by structural elements as follows:

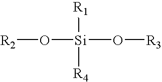

Silicone oils are high-molecular, synthetic polymeric compounds, in which silicon atoms are linearly linked and/or crosslinked via oxygen atoms, and the residual valences of the silicon are saturated by hydrocarbon residues (in most cases methyl-, to a lesser extent ethyl-, propyl-, phenyl groups, and others).

Linear silicones with a plurality of siloxyl units that are advantageous to use in accordance with the invention, are generally characterized by structural elements, such as 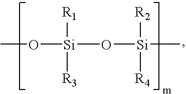
where the silicon atoms can be substituted with the same or different alkyl residues and/or aryl residues, which are generally shown by the residues R₁-R₄ (i.e., the number of the different residues is not necessarily limited to as many as four); m may have values from 2 to 200,000.

Systematically, the linear silicone oils are called polyorganosiloxanes; the methyl-substituted polyorganosiloxanes, which represent in terms of quantity the most important compounds of this group and are characterized by the following structural formula 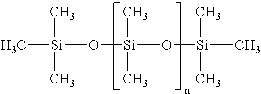
are also named polymethylsiloxanes or dimethicones (INCI). Dimethicones exist in different chain lengths or with different molecular weights. Dimethicones of different chain lengths and phenyltrimethicones are particularly advantageous linear silicone oils for the purposes of the present invention.

Particularly advantageous polyorganosiloxanes for the purposes of the present invention are furthermore, for example, dimethyl polysiloxanes [poly(dimethylsiloxanes)], which are available, for example, under the tradenames ABIL 10 to 10,000 from Th. Goldschmidt. Also advantageous are phenylmethyl polysiloxanes (INCI: phenyl dimethicones, phenyl trimethicones), cyclic silicones (octamethyl cyclotetrasiloxane or decamethyl cyclopentasiloxane), which are also named according to INCI cyclomethicones, amino-modified silicones (INCI: amodimethicones), and silicone waxes, for example, polysiloxane-polyalkylene copolymers (INCI: stearyl dimethicone and cetyl dimethicone), and dialkoxy dimethylpolysiloxanes (stearoxy dimethicone and behenoxy stearyl dimethicone), which are available as different Abil wax types from Th. Goldschmidt.

Especially advantageous for the purposes of the present invention are also the following listed silicone oils:

| Manu- | | | Polarity |
|---|---|---|---|
| facturer | Trade Name | INCI-Name | [mN/m] |
| Wacker | Wacker Silicone Oil AK | Polydimethylsiloxane | 26.9 |
| | 100 | | |
| Wacker | Wacker Silicone Oil AK 50 | Polydimethylsiloxane | 46.5 |
| Wacker | Wacker Silicone Oil AK 35 | Polydimethylsiloxane | 42.4 |
| Wacker | Wacker Silicone Oil AK 20 | Polydimethylsiloxane | 40.9 |
| Dow | Dow Corning Fluid 245 | Cyclopentasiloxane | 32.3 |
| Corning | | | |
| Dow | Dow Corning Fluid 345 | Cyclomethicone | 28.5 |
| Corning | | | |

Cyclic silicones that are advantageous to use in accordance with the invention are generally characterized by structural elements, as follows: 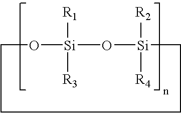
where the silicon atoms can be substituted with the same or with different alkyl residues and/or aryl residues, which are generally represented by the residues R₁-R₄ (i.e., the number of the different residues is not necessarily limited to as many as four); n may assume values from 3/2 to 20. Fractional values for n take into account that uneven numbers of siloxyl groups can be present in the cycle.

Particularly advantageous cyclic silicone oils for the purposes of the present invention are cyclomethicone, in particular cyclomethicone D5 and/or cyclomethicone D6.

Advantageous silicone oils or silicone waxes for the purposes of the present invention are cyclic and/or linear silicone oils and silicone waxes.

It is especially advantageous for the purposes of the present invention to choose a ratio of lipids to silicone oils of approximately 1:1 (generally x:y).

It is advantageous to select phenyl trimethicone as silicone oil. Likewise other silicone oils, for example, dimethicone, phenyl dimethicone, cyclomethicone (octamethyl cyclotetrasiloxane), for example, hexamethyl cyclotrisiloxane, polydimethylsiloxane, poly(methyl)phenylsiloxane) cetyl dimethicone, behenoxy dimethicone are advantageous to use for the purposes of the present invention.

Also advantageous are mixtures of cyclomethicone and isotridecyl isononanoate, as well as those of cyclomethicone and 2-ethylhexyl isostearate.

However, it is also advantageous to choose silicone oils of a constitution similar to that of the foregoing compounds, whose organic side chains are derivatized, for example, polyethoxylated and/or polyproxylated. Among those are, for example, polysiloxane-polyalkyl-polyether copolymers, such as cetyl-dimethicone-copolyol, as well as cetyl-dimethicone-copolyol (and) polyglyceryl-4-isostearate (and) hexyllaurate.

The emulsions of the invention may advantageously contain moisturizing or humectant agents (so-called moisturizers). Advantageous moisturizers for the purposes of the present invention are, for example, glycerin, lactic acid and/or lactate, in particular, sodium lactate, butylene glycol, propylene glycol, biosaccharide gum-1, glycine soya, ethylhexyloxyglycerin, pyrrolidone carboxylic acid, and urea. Furthermore, it is of special advantage to use polymeric moisturizers from the group of the water-soluble and/or water-swellable and/or water-gellable polysaccarides. Particularly advantageous are, for example, hyaluronic acid, chitosan, and/or a fucose-rich polysaccharide, which is filed in the Chemical Abstracts under the Registry Number 178463-23-5, and which can be obtained, for example, under the name Fucogel® 1000 from SOLABIA S.A.

Antioxidants are advantageously selected from the group consisting of amino acids (for example, glycine, lysine, arginine, cysteine, histidine, tyrosine, tryptophan) and derivatives thereof (as salt-, ester-, ether-, sugar-, nucleotide-, nucleoside-, peptide-, and lipid compounds); imidazoles (for example, urocanic acid) and their derivatives (as salt-, ester-, ether-, sugar-, nucleotide-, nucleoside-, peptide-, and lipid compounds); peptides, such as D,L-carnosine, D-carnosine, L-carnosine, anserine, and derivatives thereof (for example, as salt-, ester-, ether-, sugar-, thiol-, nucleotide-, nucleoside-, peptide-, and lipid compounds); carotenoids, carotene (for example, α-carotene, β-carotene, ψ-lycopene, phytoene) and derivatives thereof (for example, as salt-, ester-, ether-, sugar-, nucleotide-, nucleoside-, peptide-, and lipid compounds); chlorogenic acid and its derivates (as salt-, ester-, ether-, sugar-, thiol-, nucleotide-, nucleoside-, peptide-, and/or lipid compounds); aurothioglucose, propylethiouracil and other thiols (for example, thioredoxin, lipoic acid, glutathione, cysteine, cystine, cystamine, and glycosyl-, N-acetyl-, methyl-, ethyl-, propyl-, amyl-, butyl- and lauryl-, palmitoyl-, oleyl-, γ-linoleyl-, cholesteryl-, and glyceryl esters thereof, as well as their salts, dilauryl thiodipropionate, distearyl thiodipropionate, thiodipropionic acid and derivatives thereof (as salt-, ester-, ether-, sugar-, thiol-, nucleotide-, nucleoside-, peptide and/or lipid compounds), and sulfoximine compounds (for example, homocysteine sulfoximine, buthionine sulfones, penta-, hexa-, heptathionine sulfoximine) in very low tolerated doses (for example, pmol to μmol/kg). Furthermore, metal chelating agents (for example, apoferritin, desferral, lactoferrin, α-hydroxy fatty acids, palmitic acid, phytic acid), and derivatives thereof (as salt-, ester-, ether-, sugar-, thiol-, nucleotide-, nucleoside-, peptide and/or lipid compounds); α-hydroxy acids (for example, citric acid, lactic acid, malic acid), humic acid, bile acid, bile extracts, bilirubin, biliverdin, EDTA, EGTA and derivatives thereof; unsaturated fatty acids and derivatives thereof; unsaturated fatty acids and their derivatives (for example, γ-linolenic acid, linoleic acid, oleic acid), folic acid, and derivatives thereof; furfurylidene sorbitol and its derivatives; ubiquinone, ubiquinol, plastoquinone and their derivatives (as salt-, ester-, ether-, sugar-, thiol-, nucleotide-, nucleoside-, peptide and lipid compounds); vitamin C and derivatives (for example, ascorbyl palmitate, Mg-ascorbyl phosphate, ascorbyl acetate); tocopherols and derivatives thereof (for example, vitamin E acetate) as well as phenolic compounds and plant extracts containing same, such as, for example, flavonoids (for example, glycosylrutin, ferulic acid, caffeic acid); furfurylidene glucitol, butylhydroxytoluene, butylhydroxyanisole, nordihydroguaiac resin acid; nordihydroguaiaretic acid; trihydroxybutyrophenone and derivatives thereof (as salt-, ester-, ether-, sugar-, nucleotide-, nucleoside-, peptide and lipid compounds); uric acid and its derivatives; mannose and its derivatives (as salt-, ester-, ether-, sugar-, nucleotide-, nucleoside-, peptide and lipid compounds); zinc and its derivatives (for example, ZnO, ZnSO₄); selenium and its derivatives (for example, selenium methionine, Ebselen); stilbene and its derivatives for example, stilbene oxide, trans-stilbene oxide); and derivatives (salt-, ester-, ether-, sugar-, thiol-, nucleotide-, nucleoside, peptide-, and/or lipid compounds) of these referenced active ingredients, which are suitable according to the invention.

Further advantageous active ingredients for the purpose of the present invention are natural ingredients and/or their derivatives, such as, for example, alpha-lipoic acid, phytoene, D-biotin, coenzyme Q10, alpha-glucosylrutin, carnitine, carnosine, natural and/or synthetic isoflavonoids, creatine, creatinine, lignans, taurine, and/or beta-alanine.

Formulations of the invention, which contain, for example, known antiwrinkle agents, such as flavone glycosides (in particular alpha-glycosylrutin), coenzyme Q10, vitamin E and/or derivatives, and the like, are especially suited for prophylaxis and treatment of cosmetic or dermatological skin changes, as they occur, for example, in the case of skin ageing (such as, for example, dryness, roughness, and formation of dryness lines, itching, reduced refatting (for example, after washing), visible vascular dilations (teleangiectases, cuperosis), flaccidity and formation of wrinkles and lines, local hyperpigmentation, hypopigmentation, abnormal pigmentation (for example, age spots), increased susceptibility to mechanical stress (for example, cracking), and the like. They are furthermore suited for treating the appearance of dry and rough skin.

According to the invention, however, it is also possible and advantageous to incorporate in the preparations of the invention other pharmaceutically or dermatologically active substances, such as, for example, skin-soothing and skin-caring substances. Among those are, for example, panthenol, allantoin, tannin, antihistamines, antiphlogistic agents, glucocorticoids (for example, hydrocortisone), as well as plant ingredients, such as azulene and bisabolol, glycyrrhizin, hamamelin, and plant extracts, such as chamomile, aloe vera, hamamelis, and the root of licorice. Likewise, the vitamin D₃ analogues tacalcitol, calcipotriol, cholecalciferol, as well as calcitrol (vitamin D₃), and/or esters of the fumaric acid may be successfully included in the preparations.

The preparations of the invention may contain one or more of these active constituents (moisturizers, antioxidants, other active ingredients), each in a concentration of 0.001 to 30% by weight based on the total weight of the preparation.

It is advantageous in accordance with the invention, when the emulsions of the invention include a content of UV protective substances. For example, day creams or makeup products normally include UV-A or UV-B filter substances. Likewise UV protective substances constitute in the same way as antioxidants and, if desired, preservative, an effective protection of the preparations against deterioration. Also favorable are cosmetic and dermatological preparations, which are present in the form of a sunscreen.

Accordingly, the emulsions contain for the purposes of the present invention preferably at least one UV-A- and/or UV-B filter substance. While not necessary, the formulations may also contain one or more organic and/or inorganic pigments as UV-B filter substances, which may be present in the water- and/or oil phase.

Preferred inorganic pigments are metal oxides and/or other metal compounds that are difficult to dissolve or insoluble in water, in particular the oxides of titanium (TiO₂), zinc (ZnO), iron (for example, Fe₂O₃), zirconium (ZrO₂), silicon (SiO₂), manganese (for example, MnO), aluminum (Al₂O₃), cerium (for example (Ce₂O₃), mixed oxides of the corresponding metals, as well as mixtures of such oxides, as well as the sulfate of barium (BASO₄).

The titanium dioxide pigments may be present in the crystalline modification rutile as well as anatase. For the purposes of the present invention, they may advantageously be surface treated (“coated”). In this process, it is intended to form or maintain, for example, a hydrophilic, amphiphilic, or hydrophobic character. This surface treatment may consist in that the pigments are provided by methods known per se with a thin hydrophilic and/or hydrophobic, inorganic and/or organic coating. For the purposes of the present invention, the different surface coatings may also contain water.

The foregoing coated or uncoated titanium dioxides may also be used for the purposes of the present invention in the form of commercially available, oily or aqueous predispersions. It is possible and advantageous to add to these predispersions dispersants and/or solubilization agents.

The titanium dioxides of the invention are characterized by a primary particle size from 10 nm to 150 nm.

| | | Additional | |
|---|---|---|---|
| | | Constit- | |
| | | uents | |
| | | of the | |
| | | Predis- | |
| Trade name | Coating | persion | Manufacturer |
| MT-100TV | Aluminumhydroxide | — | Tayca Corporation |
| | stearic acid | | |
| MT-100Z | Aluminumhydroxide | — | Tayca Corporation |
| | stearic acid | | |
| MT-100F | Stearic acid | — | Tayca Corporation |
| | iron oxide | | |
| MT-500SAS | Alumina, silica | — | Tayca Corporation |
| | silicone | | |
| MT-100AQ | Silica | — | Tayca Corporation |
| | aluminumhydroxide | | |
| | alginic acid | | |
| Eusolex T-2000 | Alumina | — | Merck KgaA |
| | simethicone | | |
| Eosolex TS | Alumina, stearic | — | Merck KgaA |
| | acid | | |
| Titanium dioxide | None | — | Degussa |
| P25 | | | |
| Titanium dioxide | Octyltrimethylsilane | — | Degussa |
| T805 | | | |
| (Uvinul TiO₂) | | | |
| UV-Titanium | Alumina | — | Kemira |
| X170 | dimethicone | | |
| UV-Titan X161 | Alumina, silica | — | Kemira |
| | stearic acid | | |
| Tioveil AQ 10PG | Alumina | Water | Solaveil |
| | silica | propylene | Uniquema |
| | | glycol | |
| Mirasun TiW 60 | Alumina | Water | Rhone-Poulenc |
| | silica | | |

For the purposes of the present invention, specially preferred titanium dioxides are MT-100 Z and MT-100 TV from Tayca Corporation, Eusolex T-2000 and Eusolex TS from Merck, and titanium dioxide T 805 from Degussa.

Zinc oxides for the purposes of the present invention may also be used in the form of commercially available, oily or aqueous predispersions. In accordance with the invention, suitable zinc oxide particles and predispersions of zinc oxide particles are characterized by a primary particle size of <300 nm, and are available from the listed manufacturers under the following trade names:

| Trade name | Coating | Manufacturer |
|---|---|---|
| Z-Cote HP1 | 2% Dimethicone | BASF |
| Z-Cote | / | BASF |
| ZnO NDM | 5% Dimethicone | H&R |
| MZ 707M | 7% Dimethicone | M. Tayca Corp. |
| Nanox 500 | / | Elementis |
| ZnO Neutral | / | H&R |

Specially preferred zinc oxides for the purposes of the present invention are Z-Cote HP1 from BASF and the zinc oxide NDM from Haarmann & Reimer.

The total quantity of one or more inorganic pigments in the finished cosmetic preparation is advantageously selected from a range of 0.1% to 25% by weight, preferably 0.5% to 18% by weight.

An advantageous organic pigment for the purposes of the present invention is 2,2′-methylene-bis-(6-(2H-benzotriazole-2-yl)-4-(1,1,3,3-tetramethylbutyl)-phenol) [INCI: bisoctyltriazole], which is available from CIBA-Chemikalien GmbH under the trade name Tinosorb® M.

Advantageous UV-A filter substances for the purposes of the present invention are dibenzoylmethane derivatives, in particular 4-(ter.-butyl)-4′-methoxydibenzoylmethane (CAS No. 70356-09-1), which is sold by Givaudan under the trademark Parsol® 1789 and by Merck under the trade name Eusolex® 9020.

Further advantageous UV-A filter substances are phenylene-1,4-bis-(2-benzimidazyl)-3,3′-5,5′-tetrasulfonic acid and its salts, in particular the corresponding sodium, potassium, or triethanolammonium salts, in particular phenylene-1,4-bis(2-benzimidazyl)-3,3′-5,5′-tetrasulfonic acid-bis-sodium salt with the INCI name Bisimidazylate, which is available from Haarmann & Reimer, for example, under the trade name Neo Heliopan AP.

Also advantageous are 1,4-di(2-oxo-10-sulfo-3-bornylidene methyl)benzene and its salts (in particular the corresponding 10-sulfato compounds, in particular the corresponding sodium, potassium, or triethanolammonium salt), which is also referred to as benzene-1,4-di(oxo-3-bornylidenemethyl-10-sulfonic acid).

Further advantageous UV-A filter substances are hydroxybenzophenones, which are characterized by the following structural formula: 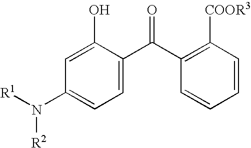
where
R¹ and R² are independently of each other hydrogen, C₁-C₂₀-alkyl, C₃-C₁₀-cycloalkyl, or C₃-C₁₀-cycloalkenyl, with the substituents R¹ and R² being able to form a 5- or 6-member ring together with the nitrogen atom to which they are linked, and
R³ is a C₁-C₂₀ alkyl residue.

A particularly advantageous hydroxybenzophenone for the purposes of the present invention is 2-(4′-diethylamino-2′-hydroxybenzoyl)benzoic acid hexyl ester (also known as: aminobenzophenone), which is available from BASF under the trade name Uvinul A Plus.

Advantageous UV filter substances for the purposes of the present invention further include so-called broadband filters, i.e. filter substances which absorb both UV-A and UV-B radiation.

Advantageous broadband filters or UV-B filter substances are, for example, bis-resorcinyltriazine derivatives with the following structure: 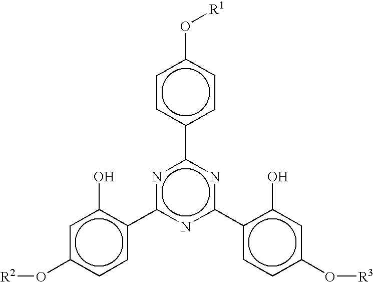
where R¹, R², and R³ are selected independently of one another from the group of branched and unbranched alkyl groups having 1 to 10 carbon atoms, or represent an individual hydrogen atom. Especially preferred is 2,4-bis{[4-(2-ethyl-hexyloxy)-2-hydroxy]phenyl}-6-(4-methoxyphenyl)-1,3,5-triazine (INCI: Aniso triazine), which is available from CIBA-Chemikalien GmbH under the trade name Tinosorb® S.

Especially advantageous preparations for the purposes of the present invention, which are characterized by a high or very high UV-A protection, preferably contain a plurality of UV-A- and broadband filters, in particular dibenzoylmethane derivatives [for example, 4-(tert.-butyl)-4′-methoxydibenzoylmethane], benzotriazole derivatives [for example, 2,2′-methylene-bis-(6-(2H-benzotriazole-2-yl)-4-(1,1,3,3-tetramethylbutyl)-phenol)], phenylene-1,4-bis(2-benzimidazyl)-3,3′-5,5′-tetrasulfonic acid and/or its salts, 1,4-d(2-oxo-10-sulfo-3-bornylidenemethyl)-benzene and/or the salts thereof, and/or 2,4-bis{[4-(2-ethyl-hexyloxy)-2-hydroxy]phenyl}-6-(4-methoxyphenyl)-1,3,5-triazine, each individually or in any combinations with one another.

Likewise other UV filter substances, which have the structural motif 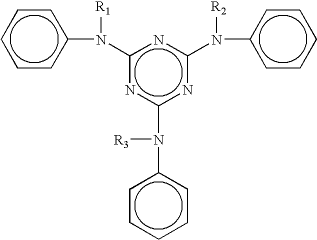
are advantageous filter substances for the purposes of the present invention, for example, s-triazine derivatives as disclosed in EP 570 838 A1, whose chemical structure is represented by the generic formula 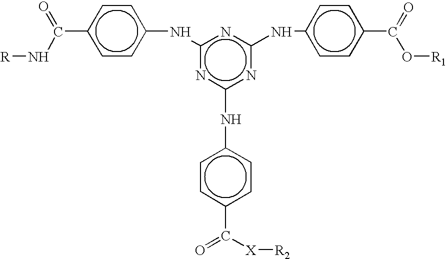
where
R is a branched or unbranched C₁₋₁₈ alkyl residue, a C₅₋₁₂ cycloalkyl residue, if need be, substituted with one of more C₁₋₄ alkyl groups,
X is an oxygen atom or an NH group, and
R₁ is a branched or unbranched C₁₋₁₈ alkyl residue, a C₅₋₁₂ cycloalkyl residue, if need be, substituted with one of more C₁₋₄ alkyl groups, or a hydrogen atom, an alkaline metal, an ammonium group, or a a group of the formula 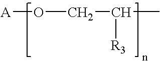
where
   A is a branched or unbranched C₁₋₁₈ alkyl residue, a C₅₋₁₂ cycloalkyl or aryl residue, if need be, substituted with one of more C₁₋₄ alkyl groups,
   R₃ is a hydrogen atom or a methyl group, and
   n is a number from 1 to 10;
R₂ is a branched or unbranched C₁₋₁₈ alkyl residue, a C₅₋₁₂ cycloalkyl residue, if need be, substituted with one of more C₁₋₄ alkyl groups, when X is the NH group, and
   a branched or unbranched C₁₋₁₈ alkyl residue, a C₅₋₁₂ cycloalkyl residue, if need be, substituted with one of more C₁₋₄ alkyl groups, or a hydrogen atom, an alkaline metal atom, an ammonium group or a group of the formula 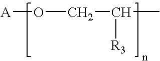
   where
   A is a branched or unbranched C₁₋₁₈ alkyl residue, a C₅₋₁₂ cycloalkyl residue, if need be, substituted with one of more C₁₋₄ alkyl groups,
   R₃ is a hydrogen atom or a methyl group, and
   n is a number from 1 to 10,
   when X is an oxygen atom.

Furthermore, a very preferred UV filter substance for the purposes of the present invention is an asymmetrically substituted s-triazine, which is also referred to in the following as dioctylbutylamidotriazone (INCI: Dioctyl butamido triazone) and can be obtained from Sigma 3V under the trade name UVASORB HEB.

Advantageous for the purposes of the present invention is also a symmetrically substituted s-triazine, i.e., 4,4′,4″-(1,3,5-triazine-2,4,6-triyltriimino)-tris-benzoic acid-tris(2-ethylhexyl-ester), synonym: 2,4,6-tris-[anilino-(p-carbo-2′-ethyl-1′-hexyloxy)]-1,3,5-triazine (INCI: Octyl Triazone), which BASF Aktiengesellschaft markets under its trade name UVINUL® T 150.

Likewise EP 775 698 discloses bis-resorcinyl triazine derivatives that are to be used with preference and whose chemical structure is represented by the generic formula 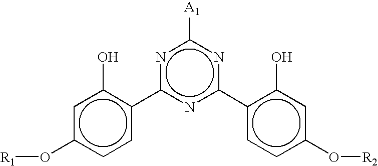
where R₁, R₂, and A₁ represent a great variety of organic residues.

Also advantageous for the purposes of the present invention are 2,4-bis-{[4-(3-sulfonato)-2-hydroxy-propyloxy)-2-hydroxy]-phenyl}-6-(4-methoxyphenyl)-1,3,5-triazine sodium salt, 2,4-bis-{[4-(3-(2-propyloxy)-2-hydroxy-propyloxy)-2-hydroxy]-phenyl}-6-(4-methoxyphenyl)-1,3,5-triazine, 2,4-bis-{[4-(2-ethyl-hexyloxy)-2-hydroxy]-phenyl}-6-[4-(2-methoxy-ethyl-carboxyl)-phenylamino]-1,3,5-triazine, 2,4-bis-{[4-(3-(2-propyloxy)-2-hydroxy-propyloxy)-2-hydroxy]-phenyl}-6-[4-(2-ethyl-carboxyl)-phenylamino]-1,3,5-triazine, 2,4-bis-{[4-(2-ethyl-hexyloxy)-2-hydroxy]-phenyl}-6-(1-methyl-pyrrol-2-yl)-1,3,5-triazine, 2,4-bis-{[4-tris(trimethylsiloxy-silylpropyloxy)-2-hydroxy]-phenyl}-6-(4-methoxyphenyl)-1,3,5-triazine, 2,4-bis-{[4-(2″-methylpropenyloxy)-2-hydroxy]-phenyl}-6-(4-methoxyphenyl)-1,3,5-triazine, and 2,4-bis-{[4-(1′,1′,1′,3′,5′,5′,5′-heptamethylsiloxy-2″-methyl-propyloxy)-2-hydroxy]-phenyl}-6-(4-methoxyphenyl)-1,3,5-triazine.

An advantageous broadband filter for the purposes of the present invention is 2,2′-methylene-bis-(6-(2H-benzotriazole-2-yl)-4-(1,1,3,3-tetramethylbutyl)-phenol), which is available from CIBA-Chemikalien GmbH under the trade name Tinosorb® M.

A further advantageous broadband filter for the purposes of the present invention is 2-(2H-benzo-triazole-2-yl)-4-methyl-6-[2-methyl-3-[1,3,3,3-tetramethyl-1-[(trimethylsilyl)oxy]disiloxanyl]propyl]-phenol (CAS-Nr.: 155633-54-8) under the INCI name Drometrizole Trisiloxane.

The UV-B and/or broadband filters may be oil-soluble or water-soluble. Advantageous oil-soluble UV-B filter and/or broadband filter substances are, for example:
derivatives of 3-benzylidene camphor, preferably 3-(4-methylbenzylidene) camphor, 3-benzylidene camphor;
derivatives of 4-aminobenzoic acid, preferably 2-(ethylhexyl)4-dimethylamino-benzoate, amyl 4-(dimethylamino)-benzoate;
2,4,6-trianilino-(p-carbo-2′-ethyl-1′-hexyloxy)-1,3,5-triazine;
esters of benzalmalonic acid, preferably di(2-ethylhexyl) 4-methoxybenzalmalonate;
esters of cinnamic acid, preferably (2-ethylhexyl)4-methoxy cinnamate, isopentyl 4-methoxy cinnamate;
derivatives of benzophenone, preferably 2-hydroxy-4-methoxybenzophenone, 2-hydroxy-4-methoxy-4′-methylbenzophenone, 2,2′-dihydroxy-4-methoxybenzophenone; as well as
polymer-linked UV filters.

Advantageous water-soluble UV-B and/or broadband filters are, for example:
salts of 2-phenylbenzimidazole-5-sulfonic acid, such as its sodium-, potassium-, or its triethanol ammonium salt, as well as the sulfonic acid itself; and
sulfonic acid derivatives of 3-benzylidene camphor, such as, for example, 4-(2-oxo-3-bornylidene-methyl)benzenesulfonic acid, 2-methyl-5-(2-oxo-3-bornylidenemethyl)sulfonic acid and their salts.

For the purposes of the present invention, especially advantageous liquid UV filter substances that can be used at room temperature are homomenthyl salicylate (INCI: homosalate), 2-ethylhexyl-2-hydroxy benzoate (2-ethylhexyl salicylate, octyl salicylate, INCI: octyl salicylate), 4-isopropylbenzyl salicylate und esters of the cinnamic acid, preferably 4-methoxy cinnamic acid(2-ethylhexyl)ester (2-ethylhexyl-4-methoxycinnamate, INCI: octyl methoxycinnamate), and 4-methoxycinnamic acid-isopentylester (isopentyl-4-methoxycinnamate, INCI: isoamyl p-methoxycinnamate), 3-(4-(2,2-bis-ethoxycarbonylvinyl)-phenoxy)propenyl)-methoxysiloxane/dimethylsiloxane copolymer (INCI: dimethicodiethylbenzalmalonate), which is available, for example, under the trade name Parsol® SLX from Hoffman La Roche.

A further light protective filter substance that is advantageous to use in accordance with the invention, is ethylhexyl-2-cyano-3,3-diphenylacrylate (octocrylene), which is available from BASF under the name Uvinul® N539.

It can also be of considerable advantage to use polymer-linked and polymeric UV filter substances in preparations according to the present invention, in particular such as are disclosed in WO-A-92/20690.

The list of the specified UV filters that can be used for the purposes of the present invention, is naturally not intended to be limiting.

Advantageously, the preparations of the invention contain the substances, which absorb UV radiation in the UV-A and/or UV-B range, where the total amount of the filter substances is, for example, from 0.1% to 30% by weight, preferably 0.5% to 20% by weight, in particular 1.0% to 15.0% by weight based on the total weight of the preparations, for the purpose of making available cosmetic preparations, which protect hair or skin against the entire range of the ultraviolet radiation. They can also serve as sunscreens for the hair or skin.

According to the invention, the emulsion of the invention may advantageously contain microparticles, whose an average particle diameter is between 1 nm and 200 nm, very advantageously between 5 nm and 100 nm.

It is furthermore advantageous to choose the concentration of all particles greater than 0.1% by weight, very advantageously between 0.1% and 30% by weight based on the total weight of the preparations.

For the present invention it is essentially irrelevant, in which of the possibly naturally occurring modifications the particles are present.

It is preferred to use untreated, almost pure pigment particles, in particular those, which can also be used as dyes in the food industry and/or as absorbers of UV radiation in sunscreens. Advantageous are, for example, the zinc oxide pigments as are available from Merck, as well as those, which can be obtained under the trade names Zinc Oxide neutral from Haarmann & Reimer or NanoX from the Harcos Chemical Group.

Also advantageous are pigments, which are made water-repellent (“coated”) on their surface. This surface treatment may consist in providing the pigments with a thin hydrophobic coating by methods known per se.

Especially advantageous are TiO₂ pigments, for example, those which are coated with aluminum stearate and can be obtained under the trade name MT 100 T from TAYCA.

A further advantageous coating of the inorganic pigments consists of dimethyl polysiloxane (also: dimethicone), a mixture of fully methylated, linear siloxane polymers, which are terminally blocked with trimethylsiloxy units. Particularly advantageous for the purposes of the present invention are zinc oxide pigments, which are coated in this way.

Advantagous is also a coating of the inorganic pigments with a mixture of dimethyl polysiloxane, in particular dimethyl polysiloxane with an average chain length of 200 to 350 dimethyl polysiloxane units, and silica gel, which is also known as simethicone. It is in particular of advantage, when the inorganic pigments are coated in addition with aluminum hydroxide or hydrous aluminum oxide (also: Alumina, CAS No. 1333-84-2). Particularly advantageous are titanium dioxides, which are coated with simethicone and alumina. In this instance, the coating may also contain water. An example therefor is titanium dioxide that can be obtained from Merck under the tradename Eusolex T2000.

Advantageous for the purposes of the present invention is furthermore the use of a mixture of different pigment types both within a crystal, for example, as a ferrous mixed oxide, and by combining a plurality of pigment types within a preparation.

Preferred are furthermore boron nitride particles, for example boron nitrides of the following list:

| Trade Name | Available from |
|---|---|
| Boron Nitride Powder | Advanced Ceramics |
| Boron Nitride Powder | Sintec Keramic |
| Ceran Blanche | Kawasaki |
| HCST Boron Nitride | Stark |
| Très BN ® | Carborundum |
| Wacker-Boron Nitride | Wacker-Chemie |
| BNP | |

Wacker-Boron Nitride BNP Wacker-Chemie

It is advantageous to choose the average particle diameter of the used boron nitride particles smaller than 20 μm, very advantageously smaller than 15 μm.

Advantageous are furthermore the boron nitride particles that are available from Carborundum under the trade name Très BN® UHP 1106, and which are treated with dimethicone.

Advantageous is furthermore a coating of the boron nitride particles with polymethylhydrogensiloxane, a linear polysiloxane also known as methicone. Advantageous, methicone-treated boron nitride particles are, for example, those which are available from Carborundum under the trade name Très BN® UHP 1107.

Further advantageous pigments are microfine polymer particles.

Advantageous microfine polymer particles are for the purposes of the present invention, for example, polycarbonate, polyether, polyethylene, polypropylene, polyvinylchloride, polystyrene, polyamide, and more of the like.

Advantageous in accordance with the invention are, for example, microfine polyamide particles, which are available under the trade name SP-500 from TORAY. Also advantageous are particles of polyamide 6 (also: Nylon 6) or polyamide 12 (also: Nylon 12). Polyamide 6 is the polyamide produced from ε-aminocaproic acid (6-aminohexanoic acid) or ε-caprolactam [poly(ε-caprolactam)], and polyamide 12 is a poly(ε-laurinlactam) from ε-laurinlactam. Advantageous for the purposes of the present invention are, for example, Orgasol® 1002 (polyamide 6) and Orgasol® 2002 (polyamide 12) from ELF ATOCHEM.

While not required, it is particularly advantageous to coat the used microfine polymer particles on their surface. This surface treatment may consist in providing the pigments with a thin hydrophilic coating by methods known per se. Advantageous coatings consist, for example, of TiO₂, ZrO₂, or also other polymers.

Particularly advantageous microfine polymer particles for the purposes of the present invention are furthermore available by the method for providing hydrophobic polymer particles with a hydrophilic coating, as is disclosed in U.S. Pat. No. 4,898,913.

It is advantageous to select the average particle diameter of the used microfine polymer particles smaller than 100 μm, very advantageously smaller than 50 μm. In this connection, the form (platelets, rodlets, pellets), in which the used polymers are present, does essentially not matter.

It is furthermore advantageous to use modified polysaccharides.

Modified polysaccharides for the purposes of the present invention can be obtained, for example, by converting starch with mono-, bi-, or polyfunctional reagents or oxidation agents in reactions that largely proceed in a polymer-analogous way.

Reactions of this type are largely based on transforming the hydroxy groups of polyglucans by etherification, esterification, or selective oxidation. In this process, for example, so-called starch ethers and starch esters of the general structural formula develop 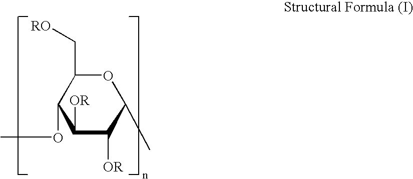
where R can be, for example, a hydrogen and/or an alkyl, and/or an aryl residue (in the case of starch ether), or a hydrogen, and/or an organic, and/or an inorganic acid residue (in the case of starch ester). Starch ethers and starch esters are advantageous modified polysaccharides for the purposes of the present invention.

Especially advantageous starch ethers are, for example, those which can be obtained by etherifying starch with tetramethylol acetylene diurea, and which are referred to as Amylym non mucilaginosum (nonswelling starch).

Especially advantageous are also starch esters and their salts, for example, the sodium- and/or aluminum salts of low-substituted half-esters of the starch, in particular sodium starch n-octenyl succinate of the structural formula (I), where R is characterized by the following structure 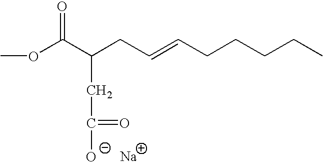
and which is available, for example, under the trade name Amiogum® 23 from CERESTAR, as well as aluminum starch octenyl succinates, in particular those which are available under the trade names Dri Flo® Elite LL, and Dri Flo® PC from CERESTAR.

It is advantageous to choose the average particle diameter of the used modified polysaccharides smaller than 20 μm, very advantageously smaller than 15 μm.

The list of the referenced modified polysaccharides is naturally not intended to be limiting. Modified polysaccharides for the purposes of the present invention are available in numerous ways of both chemical and physical nature, which are known per se.

According to the invention, the compositions may contain besides the foregoing substances, additional substances as are customary in cosmetics, for example, perfume, dyes, antimicrobial agents, refatting agents, complexing and sequestering agents, pearlescent agents, plant extracts, vitamins, active agents, preservatives, bactericides, surfactants, pigments, which have a coloring effect, thickeners, softening, moisturizing, and/or humectant substances, or other common constituents of a cosmetic or dermatological formulation, such as alcohols, polyols, polymers, foam stabilizers, electrolytes, organic solvents, or silicone derivatives.

In accordance with the invention, the preparation of the invention may advantageously contain one or more preservatives. Advantageous preservatives for the purposes of the present invention are, for example, formaldehyde donors (such as, for example, DMDM hydantoin, which is available under the trade name Glydant® from Lonza), iodopropyl butylcarbamates (for example, those which are available under the trade names Glycacil-S from Lonza and/or Dekaben LMB from Jan Dekker), parabens (i.e., alkyl esters of the p-hydroxybenzoic acid, such as methyl-, ethyl-, propyl-, and/or butylparaben), phenoxyethanol, ethanol, benzoic acid, and more of the like. Normally, the preservation system furthermore comprises in accordance with the invention advantageously preservative auxiliaries, such as, for example octoxyglycerin, glycine soya, etc. The following table gives an overview of some preservatives that are advantageous in accordance with the invention.

| | |
|---|---|
| E 200 | Sorbic acid |
| E 201 | Sodium sorbate |
| E 202 | Potassium sorbate |
| E 203 | Calcium sorbate |
| E 210 | Benzoic acid |
| E 211 | Sodium benzoate |
| E 212 | Potassium benzoate |
| E 213 | Calcium benzoate |
| E 214 | Ethyl p-hydroxybenzoate |
| E 215 | Ethyl p-hydroxybenzoate Na |
| | salt |
| E 216 | n-Propyl p-hydroxybenzoate |
| E 217 | n-Propyl p-hydroxybenzoate Na |
| | salt |
| E 218 | Methyl p-hydroxybenzoate |
| E 219 | Methyl p-hydroxybenzoate Na |
| | salt |
| E 220 | Sulfur dioxide |
| E 221 | Sodium sulfite |
| E 222 | Sodium hydrogensulfite |
| E 223 | Sodium disulfite |
| E 224 | Potassium disulfite |
| E 226 | Calcium sulfite |
| E 227 | Calcium hydrogensulfite |
| E 228 | Potassium hydrogensulfite |
| E 230 | Biphenyl (diphenyl) |
| E 231 | Orthophenylphenol |
| E 232 | Sodium |
| | orthophenylphenoxide |
| E 233 | Thiabendazole |
| E 235 | Natamycin |
| E 236 | Formic acid |
| E 237 | Sodium formate |
| E 238 | Calcium formate |
| E 239 | Hexamethylenetetramine |
| E 249 | Potassium nitrate |
| E 250 | Sodium nitrite |
| E 251 | Sodium nitrate |
| E 252 | Potassium nitrate |
| E 280 | Propionic acid |
| E 281 | Sodium propionate |
| E 282 | Calcium propionate |
| E 283 | Potassium propionate |
| E 290 | Carbon dioxide |

Also suitable according to the invention are preservatives or preservative auxiliaries that are customary in cosmetics, such as dibromocyanobutane (2-bromo-2-bromomethylglutarodinitrile), 3-iodo-2-propynyl butylcarbamate, 2-bromo-2-nitropropane-1,3-diol, imidazolidinyl urea, 5-chloro-2-methyl-4-isothiazolin-3-one, 2-chloroacetamide, benzalkonium chloride, and benzyl alcohol.

According to the invention, it is especially preferred to use as preservatives benzoic acid, and/or salicylic acid, and/or their derivatives or salts.

According to the invention, it is advantageous that one of the two partial preparations or both partial preparations contain one or more preservatives in a concentration of 2% by weight or less than 2% by weight, preferably 1.5% by weight, or less than 1.5% by weight, and more preferably 1% by weight or less than 1% by weight, each based on the total weight of the preparation A or B.

The preparations of the invention contain in an advantageous manner one or more conditioners. Preferred conditioners in accordance with the invention are, for example, all compounds which are listed in the International Cosmetic Ingredient Dictionary and Handbook (Volume 4, Publishers: R. C. Pepe, J. A. Wenninger, G. N. McEwen, The Cosmetic, Toiletry, and Fragrance Association, 9^{th} edition, 2002) in Section 4 under the keywords Hair Conditioning Agents, Humectants, Skin-Conditioning Agents, Skin-Conditioning Agents-Emollient, Skin-Conditioning Agents-Humectant, Skin-Conditioning Agents-Miscellaneous, Skin-Conditioning Agents-Occlusive, and Skin Protectants, as well as all compounds listed in EP 0 934 956 (pp. 11-13) under water soluble conditioning agent and oil soluble conditioning agent. A portion of these compounds is listed under the constituents of the aqueous phase and the oil phase in particular. Further advantageous conditioners in accordance with the invention are, for example, compounds that are called polyquaternium in accordance with the International Nomenclature for Cosmetic Ingredients (INCI), in particular Polyquaternium-1 to Polyquaternium-56).

A particularly preferred embodiment of the present invention are textiles, which are impregnated with an emulsion of the invention. According to the invention, it is preferred that the textile be present in the form of a nonwoven fabric, in particular in the form of a wipe or “pad.”

According to the invention, it is preferred to use textiles, which consist of a nonwoven fabric, in particular water-jet-bonded and/or water-jet-embossed nonwovens.

The textiles of the invention may have a smooth or also a structured surface (for example, be napped or perforated). Preferred in accordance with the invention are surface-structured textiles.

Textiles of this type may have macro embossed surfaces of any desired pattern. The selection to be made depends on the one hand on the impregnation being applied, and on the other hand on the field of application, in which the subsequent textile is to be used.

If embossed nonwovens are to be used, large cavities on the nonwoven surface and in the nonwoven fabric will facilitate absorption of dirt and impurities, when the impregnated wipe is moved over the skin. In comparison with smooth wipes the cleaning effect can be increased by a multiple.

It has been found advantageous for the wipe, when same has a weight of 35 to 120 g/m², preferably 40 to 60 g/m² (measured at 20° C.±2° C. and at a humidity of the room air of 65%±5% for 24 hours).

The average thickness of the nonwoven is preferably from 0.4 mm to 2 mm, in particular 0.6 mm to 1.2 mm (measured by the ERT method 30.5-99).

As basic materials for the nonwoven fabric of the textile, it is possible to use besides the fiber materials of the invention in general all organic an inorganic fiber materials on natural and synthetic basis. Examples include cellulose, jute, hemp, sisal, silk, wool, polypropylene, polyethylene therephthalate (PET), aramid, nylon, polyvinyl derivatives, polyurethane, polylactide, polyhydroxy alkanoate, cellulose ester and/or polyethylene, as well as mineral fibers, such as glass fibers or carbon fibers. The present invention, however, is not limited to the referenced materials. It is also possible to use a plurality of further fibers for constructing the nonwoven. It is especially advantageous for the purposes of the present invention that the used fibers are water-insoluble.

Moreover, the fibers may also be dyed to be able to highlight and/or enhance the optical attractiveness of the nonwoven. The fibers may contain in addition UV stabilizers and/or preservatives.

The fibers used for producing the fabric preferably have a water absorption rate of more than >20 mm/[10 min.] (measured by the EDANA test 10.2-96).

Furthermore, the fibers used for producing the fabric preferably have a water absorption capacity of more than >9 g/g (measured by the EDANA test 10.2-96).

Advantageous fabrics for the purposes of the present invention have a tensile strength (as measured by the ERT method 20.2-89), in particular

| | | [N/50 mm] |
|---|---|---|
| in the dry state | machine direction | >70, preferably >80 |
| | cross direction | >28, preferably >30 |
| in the impregnated | machine direction | >50, preferably >60 |
| state | cross direction | >24, preferably >30 |

The elongation of the advantageous fabric (as measured by the ERT method 20.2-89) is preferably

| | | |
|---|---|---|
| in the dry state | machine direction | 45 +/− 15% |
| | cross direction | 110 +/− 20% |
| in the impregnated | machine direction | 45 +/− 15% |
| state | cross direction | 90 +/− 20% |

According to the invention, the degree of impregnation of the cleansing article, i.e., the weight ratio of the cosmetic preparation (=liquid impregnation) and the textile is advantageously from 2.1 to 4.0, preferably 2.4 to 3.7, and more preferably 2.7 to 3.4.

In an advantageous embodiment of the nonwoven, the fibers consist of a blend of approximately 70% rayon and 30% PET.

According to the invention, a textile is preferred, which is produced from a blend of rayon fibers, polyester fibers, and/or cotton fibers.

It is especially advantageous for the purposes of the present invention to produce the textile fabric of the textile according to the invention from cotton fibers in a range from 1-30% by weight, rayon fibers in a range from 9-80% by weight, and polyester in a range from 19-90%, each based on the total weight of the textile.

In a very particularly preferred manner, the textile of the invention has on its surface a cotton content of up to 30% by weight and in its interior a cotton content of up to 10% by weight, each based on the total weight of the textile.

In accordance with the invention, the degree of impregnation is advantageously between 2 and 4. Preferred by the invention is a range between 2.8 and 3.4. In this connection, a degree of impregnation of 2.8 means that 1 g textile contains 2.8 g impregnation medium.

In accordance with the invention, however, it is also advantageous to use the emulsion as sprayable emulsion. In such a case, the emulsion of the invention can be applied with the aid of a pressure gas from an aerosol can or with the aid of a pump from a pump spray applicator.

In accordance with the invention, the emulsion of the invention, or a textile, or spray applicator is used for cleansing the skin, in particular the skin of the face.

The following examples are intended to describe in greater detail the compositions according to the invention, without however intending to limit the invention to these examples. The numerical values in the example denote weight percentages based on the total weight of the respective preparations.

| | | | (C) |
|---|---|---|---|
| | (A) m[%] | (B) m[%] | m[%] |
| Acrylates/C10-30 Alkyl Acrylate | 0.1 | 0.1 | |
| Crosspolymer | | | |
| Carbomer | | | 0.15 |
| Cetearyl isononanoate | 4.0 | | 2.0 |
| Dimethicone | | 5.0 | 3.0 |
| Fragrance | 0.2 | 0.4 | 0.3 |
| Glycerin | 4.0 | 7.0 | 3.0 |
| Glyceryl stearate citrate | 1.0 | | |
| Isohexadecane | 3.0 | 3.3 | 4.5 |
| Isopropyl isostearate | 2.0 | | 2.0 |
| Isopropyl palmitate | | 2.5 | |
| Methylparaben | 0.25 | 0.2 | 0.30 |
| Phenoxyethanol | 0.5 | 0.5 | 0.2 |
| Polyglyceryl-3 methylglucose distearate | | 0.4 | |
| Propylparaben | 0.15 | 0.15 | 0.15 |
| Triceteareth-4 phosphate | | | 1.0 |
| Sodium hydroxide | qs | qs | qs |
| Water | ad 100 | ad 100 | ad 100 |

Use of the media for impregnating fabric, rayon/polyester 10:90 to 90:10, 40 g/m² to 80 g/m², degree of impregnation 3.2

1. A cosmetic or dermatological oil-in-water (O/W) emulsion, comprising:
a) one or more emulsifiers selected from the group consisting of glyceryl stearate citrate, polyglycerol methyl glucose distearate, and triceteareth-4-phosphate, and
b) one or more polymeric stabilizers selected from the group consisting of homopolymers of acrylic acid, copolymers of acrylic acid, homopolymers of acrylic acid derivatives, and copolymers of acrylic acid derivatives,
wherein the total concentration of the sum of one or more emulsifiers a) and one or more polymeric stabilizers b) is between 0.01% and 1.5% by weight based on the total weight of the emulsion, the emulsion is free from fatty alcohols with a chain length of 12 to 20 carbon atoms, and the emulsion has a viscosity of 10 to 1500 mPa·s.

2. The emulsion as claimed in claim 1, wherein said one or more emulsifiers includes glyceryl stearate citrate.

3. The emulsion as claimed in claim 1, wherein said one or more emulsifiers includes polyglycerol methyl glucose distearate.

4. The emulsion as claimed in claim 1, wherein said one or more emulsifiers includes triceteareth-4-phosphate.

5. The emulsion as claimed in claim 1, wherein said one or more polymeric stabilizers includes acrylate/C₁₀-C₃₀alkyl acrylate copolymer.

6. The emulsion as claimed in claim 1, wherein the concentration of the polymeric stabilizer is 0.001% to 0.1% by weight based on the total weight of the preparation.

7. A cosmetic or dermatological product comprising:
a textile, and
a cosmetic or dermatological oil-in-water emulsion with a viscosity of 10 to 1500 mPa·s, said emulsion comprising:
a) one or more emulsifiers selected from the group consisting of glyceryl stearate citrate, polyglycerol methyl glucose distearate, and triceteareth-4-phosphate, and
b) one or more polymeric stabilizers selected from the group consisting of homopolymers of acrylic acid, copolymers of acrylic acid, homopolymers of acrylic acid derivatives, and copolymers of acrylic acid derivatives,
wherein the total concentration of the sum of one or more emulsifiers a) and one or more polymeric stabilizers b) in said emulsion is between 0.01% and 1.5% by weight based on the total weight of the emulsion,
wherein said emulsion is free from fatty alcohols with a chain length of 12 to 20 carbon atoms, and
wherein said textile is impregnated with said emulsion.

8. The product as claimed in claim 7, wherein the textile is present in the form of a nonwoven fabric.

9. The product as claimed in claim 7, wherein the textile is constructed from a blend of rayon fibers, polyester fibers, and cotton fibers.

10. A method for cleansing the skin comprising applying to the skin a cosmetic or dermatological oil-in-water emulsion with a viscosity of 10 to 1500 mPa·s, said emulsion comprising:
a) one or more emulsifiers selected from the group consisting of glyceryl stearate citrate, polyglycerol methyl glucose distearate, and triceteareth-4-phosphate, and
b) one or more polymeric stabilizers selected from the group consisting of homopolymers of acrylic acid, copolymers of acrylic acid, homopolymers of acrylic acid derivatives, and copolymers of acrylic acid derivatives,
wherein the total concentration of the sum of one or more emulsifiers a) and one or more polymeric stabilizers b) in said emulsion is between 0.01% and 1.5% by weight based on the total weight of the emulsion, and
wherein said emulsion is free from fatty alcohols with a chain length of 12 to 20 carbon atoms.

11. The emulsion as claimed in claim 1, wherein said one or more polymeric stabilizers includes polyacrylate/C₁₀-C₃₀ alkyl acrylate copolymer.

12. The emulsion as claimed in claim 1, wherein said one or more polymeric stabilizers includes acrylate/C₁₀-C₃₀ alkyl acrylate cross polymer.

13. The emulsion as claimed in claim 1, wherein the weight ratio of said one or more emulsifiers to said one or more polymeric stabilizers is from 20:1 to 1:1.

14. The product as claimed in claim 7, wherein said textile is in the form of a wipe.

15. The product as claimed in claim 7, wherein the textile is constructed from a blend of fibers including at least one type of fiber selected from the group consisting of rayon fibers, polyester fibers, and cotton fibers.

16. The product as claimed in claim 7, wherein the degree of impregnation of the textile is from 2.8 to 3.4.

17. The product as claimed in claim 7, wherein said textile comprises a surface and an interior, wherein the content of cotton fiber on the surface of the textile is up to 30% by weight, and wherein the content of cotton fiber in the interior of the textile is up to 10% by weight.

18. The product as claimed in claim 7, wherein said textile is surface-structured or embossed.

19. The method as claimed in claim 10, wherein said applying step comprises applying said emulsion to the skin with a textile impregnated with said emulsion.

20. A cosmetic or dermatological product comprising:
a spray applicator, and
the emulsion as claimed in claim 1,
wherein said emulsion is provided within said spray applicator, and
wherein said emulsion is capable of being sprayed.
